(19) United States
(12) Patent Application Publication
(10) Pub. No.: US 2025/0109097 A1
(43) Pub. Date: Apr. 3, 2025

(54) PHTHALONITRILE MONOMERS AND CURABLE COMPOSITION COMPRISING THE PHTHALONITRILE MONOMERS

(71) Applicant: CANON KABUSHIKI KAISHA, Tokyo (JP)

(72) Inventors: Fen WAN, Austin, TX (US); Weijun Liu, Cedar Park, TX (US)

(21) Appl. No.: 18/478,252

(22) Filed: Sep. 29, 2023

(51) Int. Cl. C07C 255/54; C09D 4/00; C09D 147/00

(52) U.S. Cl. CPC C07C 255/54; C09D 4/00; C09D 147/00

(57) ABSTRACT

A phthalonitrile monomer can have a structure of formula (1): (R₂)ₙ—X—(R₁)ₘ (1), wherein X is a substituted or unsubstituted aryl or aryl-alkyl; R₁

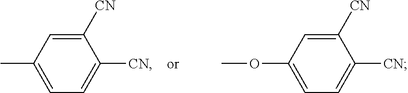

R₂ is —CH═CH₂; n is 1 to 4; and m is 1-4. A curable composition can comprise a polymerizable material, wherein the polymerizable material may include the phthalonitrile monomer of formula (1).

### FIELD OF THE DISCLOSURE

The present disclosure relates to phthalonitrile monomers and a curable composition for inkjet adaptive planarization (IAP) comprising the phtalonitrile monomers, wherein the curable composition is adapted for forming cured layers having a high thermal stability.

### BACKGROUND

Inkjet Adaptive Planarization (IAP) is a process which planarizes a surface of a substrate, e.g., a wafer containing an electric circuit, by jetting liquid drops of a photocurable composition on the surface of the substrate and bringing a flat superstrate in direct contact with the added liquid to form a flat liquid layer. The flat liquid layer is typically solidified under UV light or heat exposure, and after removal of the superstrate a planar polymeric surface is obtained, which can be subjected to subsequent processing steps, for example, baking, etching, and/or further deposition steps.

Subsequent baking of the formed cured layer is often conducted at temperatures which can reach 350° C., or 400° C. and even 450° C., and requires a high thermal stability and low shrinkage of the photo-cured layers. Most of the current UV cured resists are acrylate-based resists with a degradation starting before 300° C.

There exists a need for improved IAP materials, wherein the curable compositions have a long shelf life and which can form planar cured layers with high thermal stability during subsequent processing.

### SUMMARY

In one embodiment, a phthalonitrile monomer can have a structure of formula (1): (R₂)ₙ—X—(R₁)ₘ (1), wherein X is a substituted or unsubstituted aryl (Ar) or aryl-alkyl; R₁ is

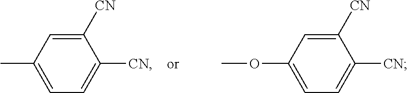

R₂ is —CH═CH₂; n is 1 to 4; and m is 1 to 4.

In one aspect, X of formula (1) of the phthalonitrile monomer can include benzyl, biphenyl, Ar₁—O—Ar₂, or Ar₁—CH₂—Ar₂.

In a certain aspect, the monomer can comprise a structure selected from formula (2), (3), or (4):

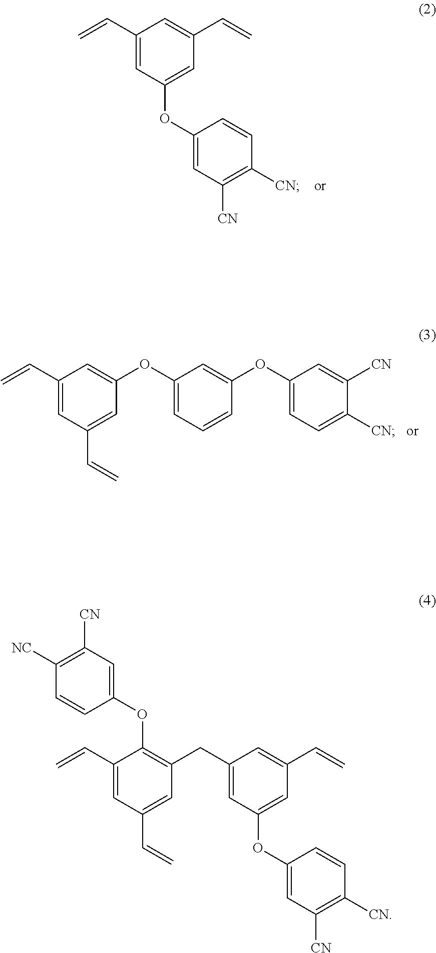

In another embodiment, a curable composition can comprise a polymerizable material, wherein the polymerizable material can comprise a phthalonitrile monomer having a structure of formula (1): (R₂)ₙ—X—(R₁)ₘ (1), wherein X is a substituted or unsubstituted aryl or aryl-alkyl; R₁ is

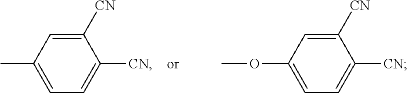

R₂ is —CH═CH₂; n is 1 to 4; and m is 1 to 4.

In one aspect of the curable composition, X of formula (1) of the phthalonitrile monomer can include benzyl, biphenyl, diphenyl ether, Ar₁—O—Ar₂, or Ar₁—CH₂—Ar₂.

In a further aspect, the phthalonitrile monomer of the curable composition can have a structure selected from formula (2), (3), or (4):

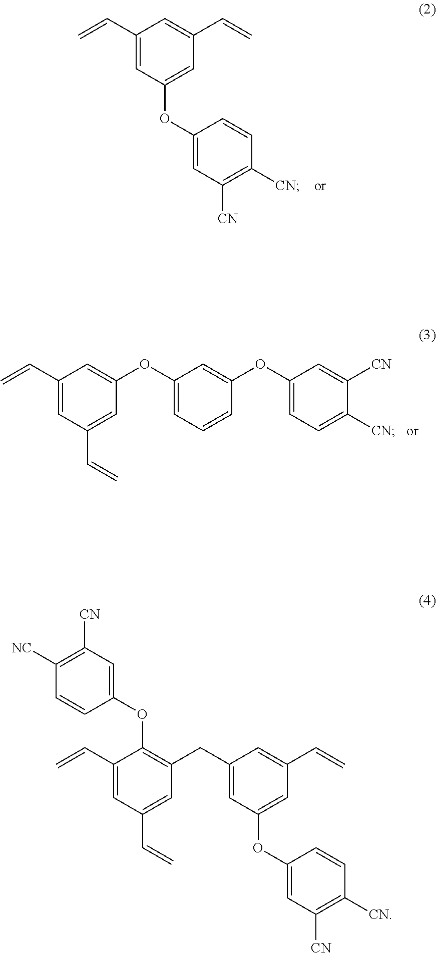

In one embodiment of the curable composition, the amount of the phthalonitrile monomer of formula (1) can be at least 1 wt % based on the total weight of the polymerizable material and not greater than 40 wt %. In a certain aspect, the amount of the phthalonitrile monomer can be at least 5 wt % and not greater than 20 wt % based on the total weight of the polymerizable material.

In another embodiment of the curable composition, the polymerizable material can comprise at least one second monomer which does not contain a phthalonitrile group.

In one aspect, the at least one second monomer of the curable composition can include an aromatic multi-functional vinylmonomer. In certain aspects, the second monomer may include a divinylbenzene monomer, a trivinylbezene monomer, a divinyldiphenyl monomer, a trivinyldiphenyl monomer, a tetravinyldiphenyl monomer, a divinyldiphenyl ether monomer, a trivinyldiphenyl ether monomer, a tetravinyldiphenyl ether monomer, a divinyldiphenylmethane monomer, a trivinyldiphenylmethane monomer, a tetravinyldiphenylmethane monomer, or any combination thereof.

In another aspect of the curable composition, the amount of the aromatic multi-functional vinylmonomer of the second monomer can be at least 50 wt % based on the total weight of the polymerizable material.

In one embodiment, the viscosity of the curable composition can be not greater than 30 mPa·s at 23° C., measured according to the Brooflield method.

In another embodiment of the curable composition, the amount of the polymerizable material can be at least 90 wt % based on a total weight of the curable composition.

In one aspect of the curable composition, the polymerizable material can have a carbon content of at least 80 percent based on the total molecular weight of the polymerizable material.

In another embodiment, a laminate can comprise a substrate and a cured layer overlying the substrate, wherein the cured layer can be formed from the above-described curable composition.

In one aspect of the laminate, the initial thermal degradation temperature T(X) of the cured layer can be at least 400° C.

In a further embodiment, a method of forming a cured layer on a substrate can comprise: applying a layer of a curable composition on the substrate, wherein the curable composition comprises a polymerizable material and an initiator, the polymerizable material comprising a phthalonitrile monomer having a structure of formula (1): (R₂)ₙ—X—(R₁)ₘ (1), wherein X is a substituted or unsubstituted aryl or aryl-alkyl, R₁ is

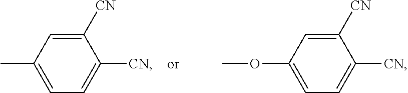

R₂ is —CH═CH₂, n is 1 to 4, and m is 1 to 4; bringing the curable composition into contact with an imprint template or a superstrate; polymerizing the curable composition with light or heat to form the cured layer; and removing the imprint template or superstrate from the cured layer.

In one aspect of the method, the initial thermal degradation temperature T(X) of the cured layer can be at least 400° C.

In yet a further embodiment, a method of manufacturing an article can comprise: forming the cured layer on the substrate according to the method of forming a cured layer described above; forming a pattern on the substrate; and processing the substrate on which the pattern has been formed in the forming; and manufacturing the article from the substrate processed in the processing.

### BRIEF DESCRIPTION OF THE DRAWINGS

Embodiments are illustrated by way of example and are not limited in the accompanying figures.

FIG. 1 includes a graph illustrating TGA curves obtained via dynamic thermogravimetry of a sample of a cured layer formed from curable composition S1 according to one embodiment and of a cured layer of a comparative sample C1.

FIG. 2 includes a graph illustrating TGA curves obtained via isothermal thermogravimetry at 350° C. and 400° C. of a sample of a cured layer formed from curable composition S1 according to one embodiment and of a cured layer of a comparative sample C1.

### DETAILED DESCRIPTION

The following description is provided to assist in understanding the teachings disclosed herein and will focus on specific implementations and embodiments of the teachings. This focus is provided to assist in describing the teachings and should not be interpreted as a limitation on the scope or applicability of the teachings.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the imprint and lithography arts.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus.

As used herein, and unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

In one embodiment, the present disclosure is directed to a phthalonitrile monomer having a structure of formula (1): (R₂)ₙ—X—(R₁)ₘ (1), wherein X is substituted or unsubstituted aryl or aryl-alkyl; R₁ is

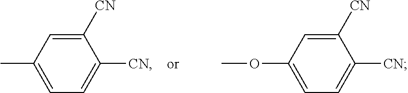

R₂ is —CH═CH₂; n is 1 to 4; and m is 1-4. R₂ can be positioned anywhere on the aryl or the aryl-alkyl group X.

In one aspect, X of formula (1) can include benzyl, biphenyl, Ar₁—O—Ar₂, or Ar₁—CH₂—Ar₂. In a particular aspect, Ar₁—O—Ar₂ may be biphenyl ether. In another aspect, Ar₁—CH₂—Ar₂ can be biphenylmethane. In a particular aspect, R₂ can include at least two vinyl groups, which can be on Ar₁ or on Ar₂, or the two or more vinyl groups of R₂ may be on Ar₁ and on Ar₂.

Non-limiting examples of phthalonitrile monomers of formula (1) can be the structures of formula (2), (3), or (4):

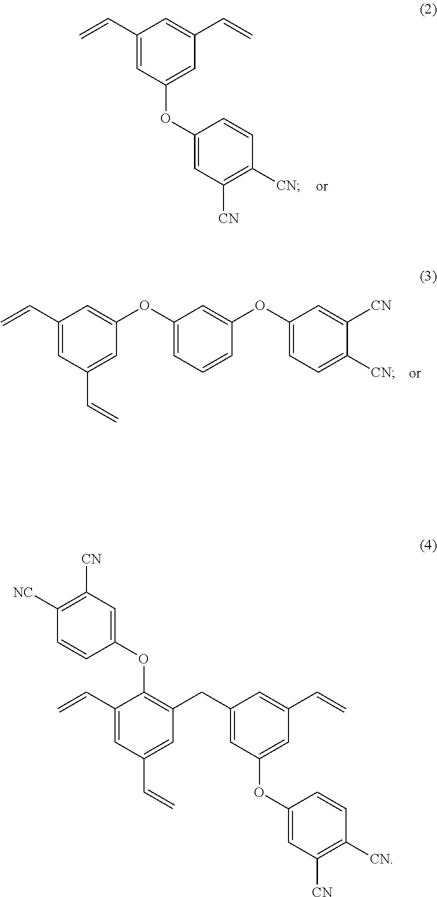 [SMILES: C=Cc1cc(C=C)cc(Oc2ccc(C#N)c(C#N)c2)c1 ; C=Cc1cc(C=C)cc(Oc2cccc(Oc3ccc(C#N)c(C#N)c3)c2)c1 ; C=Cc1cc(Cc2cc(C=C)cc(C=C)c2Oc2ccc(C#N)c(C#N)c2)cc(Oc2ccc(C#N)c(C#N)c2)c1]

The present disclosure is further directed to a curable composition comprising a polymerizable material and an initiator, wherein the polymerizable material can comprise a phthalonitrile monomer. In one embodiment, the phthalonitrile monomer of the curable composition can have a structure of the phthalonitrile monomer of formula (1) described above.

It has been surprisingly observed that a curable composition comprising a phthalonitrile monomer of formula (1) in certain combinations with one or more other polymerizable monomers can have a very high thermal stability.

As used herein the phrase “one or more other polymerizable monomers” is also synonymously called “at least one second monomer,” wherein the at least one second monomer does not contain a phthalonitrile group.

In one embodiment, the amount of the phthalonitrile monomer of formula (1) can be at least 1 wt % based on the total weight of the polymerizable material, or at least 3 wt %, or at least 5 wt %, or at least 7 wt %, or at least 10 wt %, or at least 15 wt %, or at least 20 wt %. In another aspect, the amount of the phthalonitrile monomer may be not greater than 40 wt %, or not greater than 35 wt %, or not greater than 30 wt %, or not greater than 25 wt %, or not greater than 20 wt %, or not greater than 12 wt %, or not greater than 10 wt % based on the total amount of polymerizable material. In a certain aspect, the amount of the phthalonitrile monomer may be at least 5 wt % and not greater than 20 wt % based on the total weight of the polymerizable material.

In one embodiment, the at least one second monomer can include at least one aromatic multi-functional vinylmonomer. As used herein, the term aromatic multi-functional vinylmonomer is related to a polymerizable monomer comprising at least one aromatic ring structure and at least two vinyl-groups.

In aspects, the at least one second monomer can include a vinylbenzene monomer, a divinylbenzene monomer, a trivinylbezene monomer, a divinyldiphenyl monomer, a trivinyldiphenyl monomer, a tetravinyldiphenyl monomer, a divinyldiphenyl ether monomer, a trivinyldiphenyl ether monomer, a tetravinyldiphenyl ether monomer, a divinyldiphenylmethane monomer, a trivinyldiphenylmethane monomer, a tetravinyldiphenylmethane monomer, or any combination thereof. The multi-functional vinylbenzene monomers can be further substituted by alkyl-groups, or other functional groups, for example, halogen, or amine, of hydroxyl, or carboxyl groups

Non-limiting example structures of the aromatic multi-functional vinylmonomers can be:

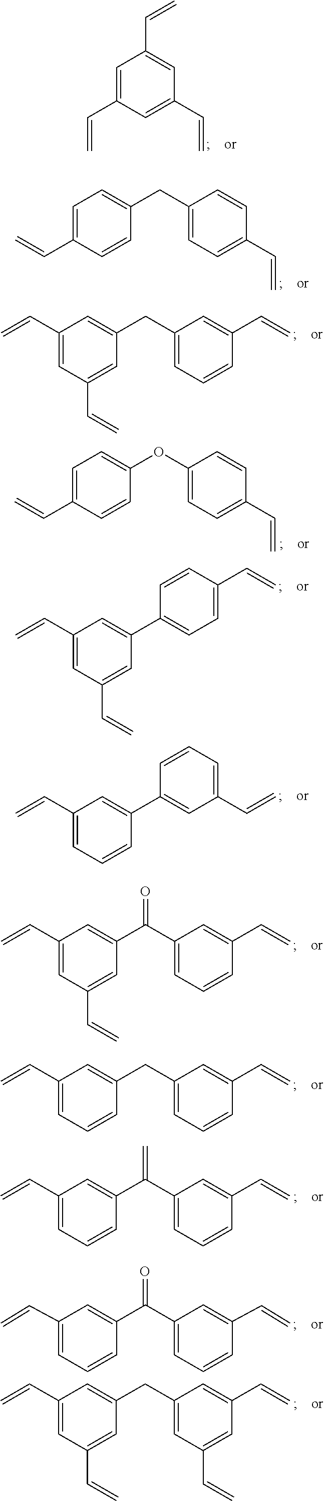

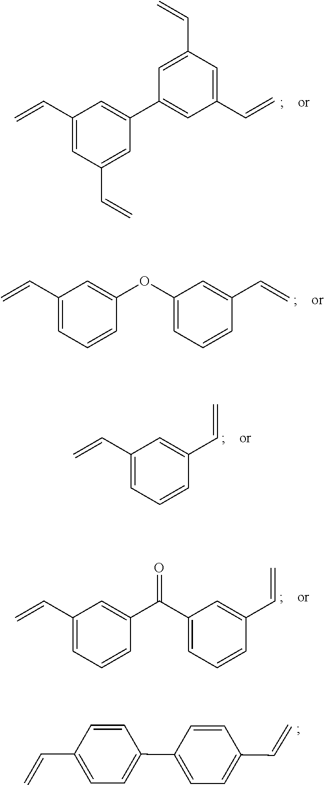

or any combination thereof.

The amount of the at least one aromatic multi-functional vinylmonomer of the at least one second monomer may be at least 50 wt % based on the total weight of the polymerizable material, or at least 65 wt %, or at least 70 wt %, or at least 75 wt %, or at least 80 wt %, or at least 85 wt %, or at least 90 wt %, or at least 95 wt %. In another aspect, the amount of the at least one aromatic multi-functional vinylmonomer may be not greater than 99 wt % based on the total weight of the polymerizable material, or not greater than 95 wt %, or not greater than 90 wt %, or not greater than 88 wt %, or not greater than 85 wt %, or not greater than 80 wt %, or not greater than 75 wt %, or not greater than 70 wt %, or not greater than 65 wt %, or not greater than 60 wt %. In a particular aspect, the amount of the at least one aromatic multi-functional vinylmonomer of the second monomer can be at least 80 wt % and not greater than 95 wt % based on the total weight of the polymerizable material.

In a certain aspect, the carbon content of the aromatic multi-functional vinylmonomer of the at least one second monomer can be at least 86% based on the total molecular weight of the aromatic multi-functional vinylmonomer, or at least 88%, or at least 90%, or at least 91%, or at least 92%, or at least 93% percent.

In a certain aspect, the at least one second monomer can consist essentially of the at least one aromatic multi-functional vinylmonomer. As used herein, the at least one second monomer consisting essentially of the aromatic multi-functional vinylmonomer means that the polymerizable material contains the at least one phthalonitrile monomer of formula (1) and the at least one second monomer, and 99 wt % of the at least one second monomer are at least one aromatic multi-functional vinylmonomer.

In a further aspect, a weight % ratio of the phthalonitrile monomer to the aromatic multi-functional vinylmonomer of the second monomer can be from 1:2 to 1:20, such as from 1:3 to 1:15, or from 1:4 to 1:12, or from 1:5 to 1:10.

The inclusion of high amounts of aromatic multi-functional vinylmonomers as the at least one second monomer together with the phthalonitrile monomer of formula (1) can have the advantage of a high carbon content of the polymerizable material, which may contribute to a high final carbon content in the formed solid layer after curing. In a particular aspect, the carbon content of the polymerizable material can be at least 72% based on the total molecular weight of the polymerizable material, or at least 74%, or at least 76%, or at least 78%, or at least 80%, or at least 82%, or at least 84%, or at least 86%, or at least 88%, or at least 90%. Assuming all of the polymerizable material is cured to form a solid layer, the carbon content of the formed solid layer after curing of the curable composition can be about the same as the carbon content of the polymerizable material.

In another embodiment, the polymerizable material of the curable composition can include a certain amount polymerizable monomers, oligomers, or polymers in addition to the phthalonitrile monomer and the at least one aromatic multi-functional vinylmonomer. Non-limiting examples of such polymerizable monomers can be, for example, mono-functional or multi-functional acrylate monomers, or a maleimide monomer.

The amount of polymerizable material in the curable composition can be at least 75 wt % based on the total weight of the photocurable composition, such as at least 80 wt %, at least 85 wt %, at least 90 wt %, or at least 95 wt %, or at least 97 wt %. In another aspect, the amount of polymerizable material may be not greater than 99.5 wt % of the curable composition, or not greater than 99 wt %, or not greater than 98 wt %, or not greater than 97 wt %, or not greater than 95 wt % based on the total weight of the curable composition. The amount of polymerizable material can be a value between any of the minimum and maximum values noted above. In a particular aspect, the amount of the polymerizable material can be at least 85 wt % and not greater than 99 wt %.

Important for the selection of monomers is the aspect of maintaining a low viscosity of the polymerizable composition before curing. In one embodiment, the viscosity of the curable composition can be not greater than 30 mPa·s, such as not greater than 25 mPa·s, not greater than 20 mPa·s, not greater than 15 mPa·s, or not greater than 10 mPa·s. In other certain embodiments, the viscosity may be at least 1 mPa·s, such as at least 3 mPa·s, such as at least 5 mPa·s, or at least 8 mPa·s. In a particularly preferred aspect, the photocurable composition can have a viscosity of not greater than 15 mPa·s. As used herein, all viscosity values relate to viscosities measured at a temperature of 23° C. with the Brookfield method using a Brookfield Viscometer.

The curable composition of the present disclosure can be designed that a cured layer formed from the curable composition may have a high thermal stability. In one aspect, an onset temperature for the thermal degradation of the photo-cured layer may be at least 350° C., or at least than 375° C., or at least 400° C., or at least 410° C., or at least 420° C., or at least 425° C., or at least 430° C., or at least 435° C., or at least 440° C., or at least 445° C. In another aspect, the initial thermal degradation temperature may be not greater than 500° C. or not greater than 480° C., or not greater than 460° C. As used herein, the onset thermal degradation temperature is also called “initial thermal degradation temperature,” T(X), and relates to the temperature in the TGA curve wherein a deflection of the curve from the almost linear plateau is first observed, shortly before the steep degradation decline of the sample.

In a certain embodiment, the curable composition of the present disclosure can be essentially free of a solvent. As used herein, if not indicated otherwise, the term solvent relates to a compound which can dissolve or disperse the polymerizable monomers but does not itself polymerize during the curing of the curable composition. The term “essentially free of a solvent” means herein an amount of solvent being not greater than 5 wt % based on the total weight of the curable composition. In a certain particular aspect, the amount of a solvent can be not greater than 3 wt %, not greater than 2 wt %, not greater than 1 wt %, or the curable composition can be free of a solvent, except for unavoidable impurities.

In another aspect, the curable composition and the present disclosure can comprise a solvent in an amount greater than 5 wt % based on the total weight of the curable composition. In a particular aspect, the amount of solvent can be at least 7 wt % based on the total weight of the curable composition, or at least 10 wt %, or at least 15 wt %, at least 20 wt %. In another aspect, the amount of solvent may be not greater than 25 wt %, or not greater than 20 wt %, or not greater than 10 wt % based on the total weight of the curable composition.

In another embodiment, in order to be suitable for IAP processing the curable composition of the present disclosure can be essentially free of particles, for example pigment particles. As used herein, being essentially free of particles means that the curable composition contains not more than 50 particles per ml having a size of 200 nm or greater.

In yet a further embodiment, the curable composition of the present disclosure may not include epoxide-group containing monomers, or epoxy-group containing oligomers, or acrylamides, or a polyurethane.

In order to initiate the curing of the curable composition if exposed to light or heat, one or more initiators can be included in the curable composition, for example one or more photoinitiators or thermal initiators. In one aspect, the amount of one or more initiators can be between 1 wt % and 6 wt % based on the total weight of the curable composition.

In a certain aspect, the curing can be also conducted by a combination of light and heat curing.

The curable composition can further contain one or more optional additives. Non- limiting examples of optional additives can be stabilizers, dispersants, solvents, surfactants, inhibitors or any combination thereof. In a certain aspect, the amount of one or more surfactants can be between 0.2 wt % and 5 wt % based on the total weight of the curable composition.

In one embodiment, the photocurable composition can be applied on a substrate to form a cured layer. As used herein, the combination of substrate and cured layer overlying the substrate is called a laminate.

The inclusion of the at least phthalonitrile monomer of formula (1) and of the at least one second monomer being an aromatic multi-functional vinylmonomer as polymerizable material can contribute to a high carbon content in the formed photo-cured layer. In one embodiment, the curable composition can be adapted that after curing the formed cured layer has a carbon content of at least 70% based on the total weight of the cured layer, or at least 72%, at least 74%, at least 76%, at least 78%, or at least 80%, or at least 82%, or at least 84%, or at least 86%, or at least 88%, or at least 90%. As used herein, the carbon content of the cured layer can be determined by elemental analysis using standard commercial analytical measurement techniques and relates to the weight % of carbon based on the total weight of the cured layer. Assuming no loss of material occurs during curing of the curable composition, the calculated carbon content of the polymerizable material can correspond to the carbon content of the cured layer.

The present disclosure is further directed to a method of forming a cured layer. The method can comprise applying a layer of the curable composition described above over a substrate, bringing the curable composition into contact with a template or superstrate; polymerizing the curable composition with light or heat to form the cured layer; and removing the template or the superstrate from the cured layer.

The substrate and the solidified cured layer may be subjected to additional processing, for example, an etching process, to transfer an image into the substrate that corresponds to the pattern in one or both of the solidified layer and/or patterned layers that are underneath the solidified layer. The substrate can be further subjected to known steps and processes for device (article) fabrication, including, for example, curing, oxidation, layer formation, deposition, doping, planarization, etching, formable material removal, dicing, bonding, and packaging, and the like.

The cured layer may be further used as an interlayer insulating film of a semiconductor device, such as LSI, system LSI, DRAM, SDRAM, RDRAM, or D-RDRAM, or as a resist film used in a semiconductor manufacturing process.

As further demonstrated in the examples, it has been surprisingly discovered that certain combinations of polymerizable monomers containing a phthalonitrile monomer of formula (1) and at least one aromatic multi-functional vinylmonomer of a second monomer in a curable composition can form curable layers with very suitable properties especially for IAP processing, such as high thermal stability and high etch resistance.

### EXAMPLES

The following non-limiting examples illustrate the concepts as described herein.

### Example 1

Synthesis of 4-(3,5-divinylphenoxy)phthalonitrile (DVBOPCN).

In a first step, 15 g of 5-hydroxyisophthalaldehyde and 17.3 g of 4-nitro-1,2-phthalonitrile were dissolved in 250 ml dimethylformamide (DMF), and 41.4 g of K₂CO₃ was added as a catalyst (see also the chemical reaction scheme below). The reaction was conducted under stirring at room temperature (23° C.) for 16 hours, and the formed intermediate product (IP) was separated by preparative chromatography with a yield of 78%.

Thereafter, 18 g of the intermediate product (IP), 106 g methyltriphenyl-phosphonium bromide (Ph₃PMeBr), and 33 g potassium tert-butoxide (t-BuOx) were dissolved in 250 ml THF and stirred for 30 minutes at 28° C. The reaction product was purified with preparative chromatography, and a white powder of DVBOPCN with a purity of 95% (as measured by LC-MS) was obtained.

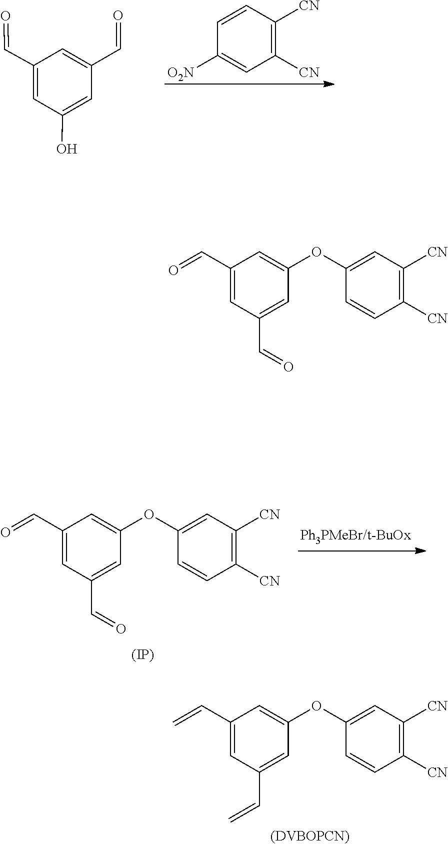

The measured H-NMR spectrum of the obtained DVBOPCN contained the following signals: δ (ppm) 5.36-5.39 (d, 2H); 5.77-5.81 (d, 2H), 6.67-6.74 (dd, 2H), 7.02, 7.02 (s, 2H), 7.25-7.36 (m, 3H), 7.73-7.75 (m, 1H).

### Example 2

### Preparing of Curable Composition

A curable composition was prepared (sample S1) by combining 10 parts DVBOPCN of the phthalonitrile monomer made in Example 1; 90 parts 3,3′-divinylbiphenyl (DVBPH); 1 part surfactant Capstone FS3100 (from The Chemours Company); 3 parts photoinitiator Irgacure 819 (from BASF) and 3 parts photoinitiator OXE02 (from BASF). The curable composition had a viscosity of 25 mPa·s at 23° C.

Furthermore, a comparative curable composition (comparative sample C1) was made containing 100 wt % DVBPH as polymerizable material, and the same type and amounts of surfactant and photoinitiators. Comparative composition C1 had a viscosity of 16.1 mPa·s at 23° C.

A summary of the compositions S1 and C1 is shown in Table 1.

TABLE 1

| Ingredient | S1 [weight parts] | C1 [weight parts] |
|---|---|---|
| DVBPH with | 90 | 100 |
| DVBOPCN | 10 | |
| FS3100 | 1 | 1 |
| OXE02 | 3 | 3 |
| Irgacure 819 | 3 | 3 |

A second set of photocurable compositions were made (samples S2 and S3) wherein 10 parts of DVBOPCN were combined with 90 parts 3,5,3′-vinyldiphenylmethane (TVPM) (Sample S2), or 90 parts DVBA (sample S3), and further including 1 part surfactant Capstone FS3100 (from The Chemours Company), 4 parts photoinitiator Irgacure 651 (from BASF) and 1.5 parts photoinitiator Omnirad 1316 (from IGM Resins). Respective comparative photocurable compositions C2 and C3 contained the same ingredients, but no DVBOPCN.

A summary of the curable compositions S2, C2, and S3, C3 of the second set is shown in Table 2.

TABLE 2

| | S2 | C2 | S3 | C3 |
|---|---|---|---|---|
| | [weight | [weight | [weight | [weight |
| Ingredient | parts] | parts] | parts] | parts] |
| TVPM | 90 | 100 | | |
| DVBA | | | 90 | 100 |
| DVBOPCN | 10 | | 10 | |
| FS3100 | 1 | 1 | 1 | 1 |
| Irgacure 651 | 4 | 4 | 4 | 4 |
| Omnirad 1316 | 1.5 | 1.5 | 1.5 | 1.5 |

### Example 3

### Thermal Shrinkage of Photo-Cured Layer After Baking Treatment

From the curable compositions summarized in Tables 1 and 2, photo-cured layers were prepared and the thermal shrinkage after exposing the cured layers to a baking treatment on a 350° C. hot-plate for 2 minutes was measured.

The photo-cured layers were prepared by applying a layer of the respective liquid curable composition on a blank fused silica wafer and photo-curing the liquid layer with light radiation having a radiation wavelength of 365 nm with total radiation dosages of 5 J, 10 J, and 20 J. The test was designed that the obtained photo-cured film (before the baking treatment) had an average thickness of about 100 nm.

The baking treatment of the photo-cured films was conducted by placing the film on a hot plate having a temperature of 375° C. and 400° C. for 2 minutes. For calculating the thermal shrinkage, the thickness of the film before and after the baking treatment was measured using a JA Woolam Spectroscopic Ellipsometer M-2000 X-210. The linear shrinkage (Sb350) was calculated according to the following equation: S_{b350}=[(Tₚ−T_{c})/Tₚ]×100%, with Tₚ being the thickness of the photo-cured film before the baking treatment, and T_{c} being the thickness of the photo-cured film after the baking.

The results of the thermal shrinkage measurements are summarized in Table 3. It can be seen that the shrinkage after baking was lower for samples S1, S2, and S3, in comparison to the respective comparative samples C1, C2, and C3, which did not contain a phthalonitrile monomer in the curable composition before curing.

TABLE 3

| | Shrinkage after baking | | | | Shrinkage after baking | | | |
|---|---|---|---|---|---|---|---|---|
| Curing | 350° C. [%] | | | | at 400° C. [%] | | | |
| Dosage | S1 | C1 | S3 | C3 | S1 | C1 | S2 | C2 |
| 5 J | | | 3.26 | 4.34 | | | 5.45 | 6.35 |
| 10 J | | | | | 8.88 | 12.32 | | |
| 20 J | 6.56 | 8.05 | | | 9.47 | 10.47 | | |

### Example 4

### Thermogravimetric Analysis.

Photo-cured films of samples S1 and C1 were made as described in Example 3, except that the thickness of the layers (films) was about 300 microns after curing, using a total UV dosage of 20J. The photo-cured layers were investigated via thermogravimetric analysis (TGA) to evaluate the thermal stability of these layers.

For the TGA measurements, a LINSEIS STA PT1000 instrument (Linseis Messgeraete GmbH, Germany) was used. All measurements were conducted under nitrogen at a rate of 5 liter per hour.

### Dynamic Thermogravimetry

For the dynamic TGA measurements, 25-35 mg of the photo-cured layer was placed in a crucible and the initial weight recorded. A reference crucible was used to monitor the weight change of the crucible due to the variation of the temperature. The sample was heated at a rate of 20° C./min and the weight loss of the sample with increasing temperature was recorded at intervals of 1 second. The relative weight percent change was calculated by using the weight loss divided by the total original weight of the sample.

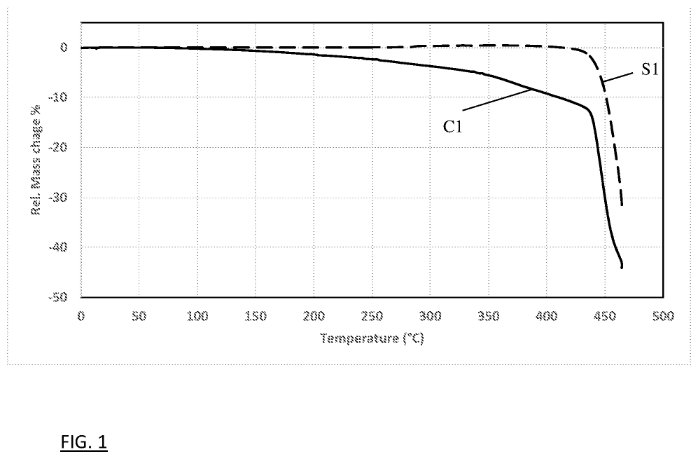

The dynamic TGA curve for the cured layers of samples S1 and C1 is shown in FIG. 1. It can be seen that the cured layer of sample S1 had a better thermal stability compared to sample C1. The initial thermal degradation temperature (T(X) for S1 was 430° C., while the initial thermal degradation temperature T(X) for C1 was 360° C.

As used herein, the thermal degradation temperature relates to the initial degradation temperature T (X), which is the temperature of the TGA curve wherein a deflection of the curve from the almost linear plateau shape is first observed, shortly before the steep decline of the mass of the sample, i.e., degradation.

### Isothermal Thermogravimetry

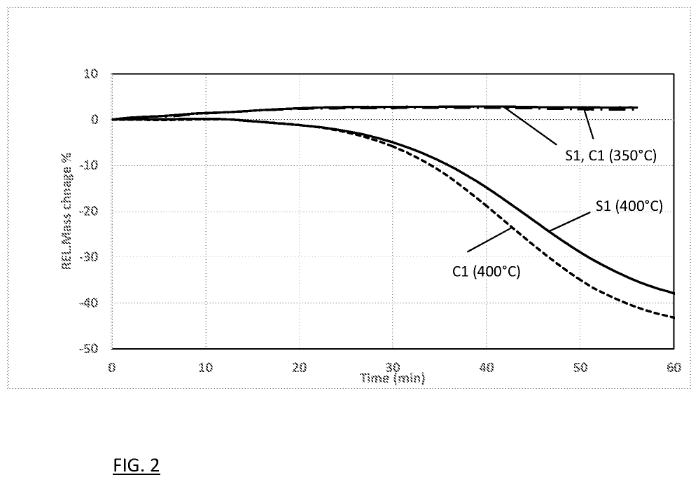

Furthermore, isothermal thermogravimetry (TGA) was conducted at constant temperatures of 350° C. and 400° C. for at least 30 minutes. The measurements were conducted with the same TGA instrument described above and also under nitrogen. For each measurement, 25-35 mg of the photo-cured sample was put in the crucible and the initial weight was recorded. The temperature was quickly raised to the aimed testing temperature (350° C. or 400° C.) (within 20 min), and when the set temperature was reached, the weight loss was recorded over the time period of 30 minutes. FIG. 2 shows the relative weight loss of photo-cured samples S1 and C1 at a constant temperature at 350° C. and at 400° C. over the time period of 30 minutes. It can be seen that at 350° C., both samples maintained stable and had no weight loss. At a temperature of 400° C., weight loss occurred with time, wherein sample S1 had a slower weight loss than comparative sample C1.

### Example 5

### Investigation of Double Bond Conversion

Fourier-transform infrared spectroscopy (FTIR) was conducted to investigate the double bond conversion after a defined curing regime of the curable composition S1 and C1 described in Example 2. For measuring the C═C conversion, the decrease of the peak at 989 cm−1 for the C═C bending peak was measured before and after the curing, while the peak for the C—H benzene ring vibration at 713 cm−1 was used as internal reference. A Thermo Nicolet 6700 FTIR with DTGS TEC detector was used to record the FTIR spectra from 4000 cm⁻¹ to 625 cm⁻¹.

For the measurement a sample amount of 0.3 to 0.5 μl was dropped on a NaCl window (25 mm×25 mm) and thereafter covered with another NaCl window of the same size. The applied UV dosage was 10 J/cm², using a UV light intensity of 100 mW/cm² at a wavelength of 365 nm. The C═C conversion was calculated by the peak ratio at 989 cm⁻¹ of the cured sample (after exposure to the curing regime) to the uncured sample (at beginning of measurement, before UV exposure).

A third IR spectrum was made after subjecting the photo-cured sample to a baking treatment at 350° C. under nitrogen on a hotplate for 3 minutes.

A summary of the calculated percent C═C conversion of the samples after UV-curing and after an additional baking treatment at 350° C. is shown in Table 4. It can be seen that the UV curing caused a C═C double-bond conversion of about 53 percent for both compositions S1 and C1. The additional baking treatment at 350° C. caused a further C═C conversion up to about 86.5% for sample S1, and 87.5% for sample C1.

TABLE 4

| Curing | C = C Conversion | C = C conversion |
|---|---|---|
| Treatment | S1 [%] | C1 [%] |
| 10 J/cm² UV | 53.6 | 53.2 |
| radiation | | |
| Baking at 350° C. | 86.5 | 87.5 |
| for 3 minutes | | |

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any sub combination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. A phthalonitrile monomer having a structure of formula (1): (R₂)ₙ—X—(R₁)ₘ (1), wherein
X is a substituted or unsubstituted aryl (Ar) or aryl-alkyl;
R₁ is 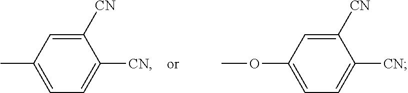
R₂ is —CH═CH₂;
n is 1 to 4; and m is 1 to 4.

2. The phthalonitrile monomer of claim 1, wherein —X— includes benzyl, biphenyl, Ar₁—O—Ar₂, or Ar₁—CH₂—Ar₂.

3. The phthalonitrile monomer of claim 2, wherein the monomer comprises a structure selected from formula (2), (3), or (4):

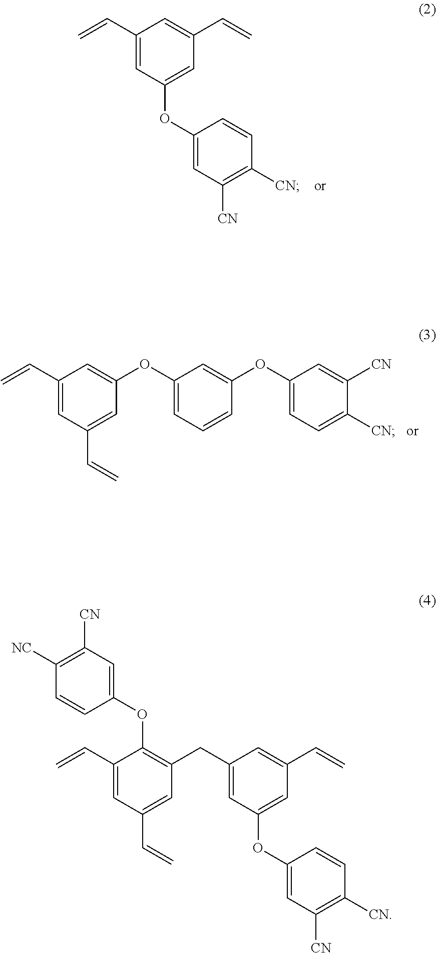

4. A curable composition comprising a polymerizable material and an initiator, wherein the polymerizable material comprises a phthalonitrile monomer having a structure of formula (1): (R₂)ₙ—X—(R₁)ₘ (1), wherein
X is a substituted or unsubstituted aryl or aryl-alkyl;
R₁ is 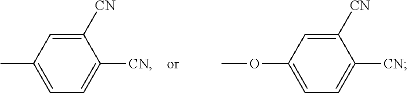
R₂ is —CH═CH₂;
n is 1 to 4; and m is 1-4.

5. The curable composition of claim 4, wherein-X-includes benzyl, biphenyl, diphenyl ether, Ar₁—O—Ar₂, or Ar₁—CH₂—Ar₂.

6. The curable composition of claim 5, wherein the phthalonitrile monomer comprises a structure selected from formula (2), (3), or (4):

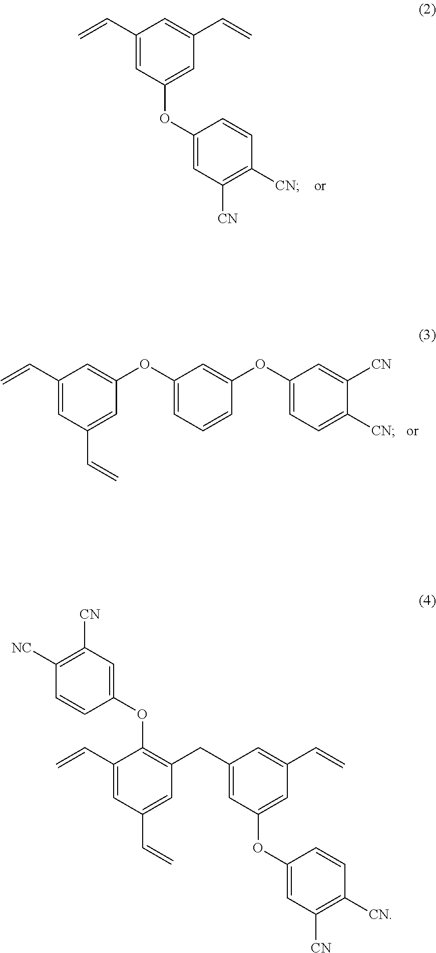

7. The curable composition of claim 4, wherein an amount of the phthalonitrile monomer is at least 1 wt % based on the total weight of the polymerizable material and not greater than 40 wt %.

8. The curable composition of claim 7, wherein the amount of the phthalonitrile monomer is at least 5 wt % and not greater than 20 wt % based on the total weight of the polymerizable material.

9. The curable composition of claim 4, wherein the polymerizable material comprises at least one second monomer which does not contain a phthalonitrile group.

10. The curable composition of claim 1, wherein the second monomer includes an aromatic multi-functional vinylmonomer.

11. The curable composition of claim 10, wherein the second monomer includes a divinylbenzene monomer, a trivinylbezene monomer, a divinyldiphenyl monomer, a trivinyldiphenyl monomer, a tetravinyldiphenyl monomer, a divinyldiphenyl ether monomer, a trivinyldiphenyl ether monomer, a tetravinyldiphenyl ether monomer, a divinyldiphenylmethane monomer, a trivinyldiphenylmethane monomer, a tetravinyldiphenylmethane monomer, or any combination thereof.

12. The curable composition of claim 10, wherein an amount of the aromatic multi-functional vinylmonomer is at least 50 wt % based on the total weight of the polymerizable material.

13. The curable composition of claim 4, wherein a viscosity of the curable composition is not greater than 30 mPa·s at 23° C.

14. The curable composition of claim 4, wherein an amount of the polymerizable material is at least 90 wt % based on a total weight of the curable composition.

15. The curable composition of claim 4, wherein the polymerizable material has a carbon content of at least 80 percent based on the total molecular weight of the polymerizable material.

16. A laminate comprising a substrate and a cured layer overlying the substrate, wherein the cured layer is formed from the curable composition of claim 4.

17. The laminate of claim 16, wherein an initial thermal degradation temperature T(X) of the cured layer is at least 400° C.

18. A method of forming a cured layer on a substrate, comprising:
applying a layer of a curable composition on the substrate, wherein the curable composition comprises a polymerizable material and an initiator, the polymerizable material comprising a phthalonitrile monomer having a structure of formula (1): (R₂)ₙ—X—(R₁)ₘ (1), wherein X is a substituted or unsubstituted aryl or aryl-alkyl; R₁ is 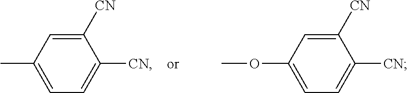
R₂ is —CH═CH₂; n is 1 to 4; and m is 1 to 4;
bringing the curable composition into contact with an imprint template or a superstrate;
polymerizing the curable composition with light or heat to form the cured layer; and
removing the imprint template or superstrate from the cured layer.

19. The method of claim 18, wherein an initial thermal degradation temperature T(X) of the cured layer is at least 400° C.

20. A method of manufacturing an article, comprising:
forming the cured layer on the substrate according to claim 18;
forming a pattern on the substrate;
processing the substrate on which the pattern has been formed in the forming; and
manufacturing the article from the substrate processed in the processing.
